(19) United States
(12) Patent Application Publication
(10) Pub. No.: US 2026/0151630 A1
(43) Pub. Date: Jun. 4, 2026

(54) LONG PULSE WIDTH SPINAL CORD STIMULATION FOR TREATING AUTONOMIC NERVE DYSFUNCTION, AND ASSOCIATED SYSTEMS AND METHODS

(71) Applicant: Nevro Corp., Redwood City, CA (US)

(72) Inventors: Kerry Bradley, Glendale, CA (US); Satinderpall Singh Pannu, Pleasanton, CA (US)

(21) Appl. No.: 19/391,605

(22) Filed: Nov. 17, 2025

Related U.S. Application Data
(63) Continuation of application No. 19/389,723, filed on Nov. 14, 2025.
(60) Provisional application No. 63/721,892, filed on Nov. 18, 2024.

(51) Int. Cl. A61N 1/36

(52) U.S. Cl. CPC A61N 1/36062; A61N 1/36067; A61N 1/36089; A61N 1/36103; A61N 1/36175; A61N 1/36182

(57) ABSTRACT

The present technology provides systems and methods for delivering electrical stimulation having relatively long pulse widths to a patient’s spinal cord region to treat autonomic nervous system dysfunction. For example, some embodiments include delivering an electrical therapy signal having individual pulses with pulse widths of between about 2 milliseconds and about 10 seconds. The electrical therapy signals may treat autonomic nervous system dysfunction by normalizing activity of the patient’s parasympathetic nervous system, normalizing activity of the patient’s sympathetic nervous system, and/or normalizing a balance of activity between the patient’s parasympathetic and sympathetic nervous systems.

### CROSS-REFERENCE TO RELATED APPLICATION(S

The present application is continuation of U.S. Patent Application No. 19/389,723, filed on November 14, 2025, which claims the benefit of priority to U.S. Provisional Application No. 63/721,892, filed on November 18, 2024, the entire contents of which are hereby incorporated by reference in its entirety for all purposes.

### TECHNICAL FIELD

The present technology is directed towards spinal cord modulation for treating patient conditions, and associated systems and methods.

### BACKGROUND

The autonomic nervous system (ANS) is largely responsible for automatically and subconsciously regulating many systems of the body, including the cardiovascular, renal, gastrointestinal, and thermoregulatory systems. By regulating these systems, the ANS can enable the body to adapt to changes in the environment, for example, changing states of stress. The autonomic nervous system includes the sympathetic system and the parasympathetic system. These two systems in many instances have opposite effects and accordingly, each one can balance the effect of the other. Autonomic nerve fibers innervate a variety of tissues, including cardiac muscle, smooth muscle, respiratory system tissues, gastrointestinal system tissues, renal system tissues, hormonal system tissues, and glands. These nerve fibers help to regulate functions associated with the foregoing tissues, including but not limited to blood pressure, respiration, blood flow, gastrointestinal functions, body temperature, bronchial dilation, blood glucose levels, metabolism, micturition and defecation, pupillary light and accommodation reflexes, and glandular secretions. Many conditions and diseases are associated with dysfunction of one or more aspects of the ANS. Accordingly, a need exists for treating autonomic nervous system dysfunction.

### BRIEF DESCRIPTION OF THE DRAWINGS

FIG. 1A is a partially schematic illustration of an implantable spinal cord modulation system positioned at a patient's spine to deliver therapeutic signals in accordance with some embodiments of the present technology.

FIG. 1B is a partially schematic, cross-sectional illustration of a patient's spine, illustrating representative locations for implanted lead bodies in accordance with some embodiments of the present technology.

FIG. 2 is a schematic illustration of a representative lead body suitable for providing modulation to a patient in accordance with several embodiments of the present technology.

FIGS. 3A-3H illustrate representative wave forms associated with therapy signals applied to patients in accordance with particular embodiments of the present technology.

### DETAILED DESCRIPTION

The present technology is generally directed to spinal cord stimulation and associated systems and methods for treating autonomic nervous system (“ANS”) dysfunction. In particular, the spinal cord stimulation described herein is expected to treat ANS dysfunction by normalizing one or more aspects of autonomic nervous system activity, such as by normalizing activity of the parasympathetic nervous system, normalizing activity of the sympathetic nervous system, and/or normalizing a balance of activity between the parasympathetic and sympathetic nervous systems. The present technology is expected to be useful in treating primary ANS dysfunction (e.g., ANS dysfunction without a known underlying cause) and secondary ANS dysfunction (e.g., ANS dysfunction caused by or otherwise associated with an underlying disease or condition, such as diabetes, high blood pressure, autoimmune disorders, etc.). In some embodiments in which secondary ANS dysfunction is being treated, the spinal cord stimulation described herein may advantageously address one or more non-ANS related aspects of the disease, in addition to treating the ANS dysfunction caused by the disease.

In many of the embodiments described herein, the spinal cord stimulation used to treat ANS dysfunction includes electrical therapy signals having relatively long pulse widths, such as pulse widths between about 2 milliseconds and about 10 seconds. In some embodiments, the electrical therapy signals further include offset high frequency pulses and/or bursts of high frequency pulses occurring during the relatively long pulse widths. For example, the therapy signal can have a base component having a non-zero amplitude and a pulse width in a pulse width range of from about 2 milliseconds to about 10 seconds, and a high frequency component including high frequency pulses having a frequency in a frequency range of from about 1.2 kHz to about 100 kHz and occurring from the non-zero amplitude of the base component. Without being bound by theory, electrical therapy signals in accordance with the present technology are expected to advantageously treat the patient’s ANS dysfunction.

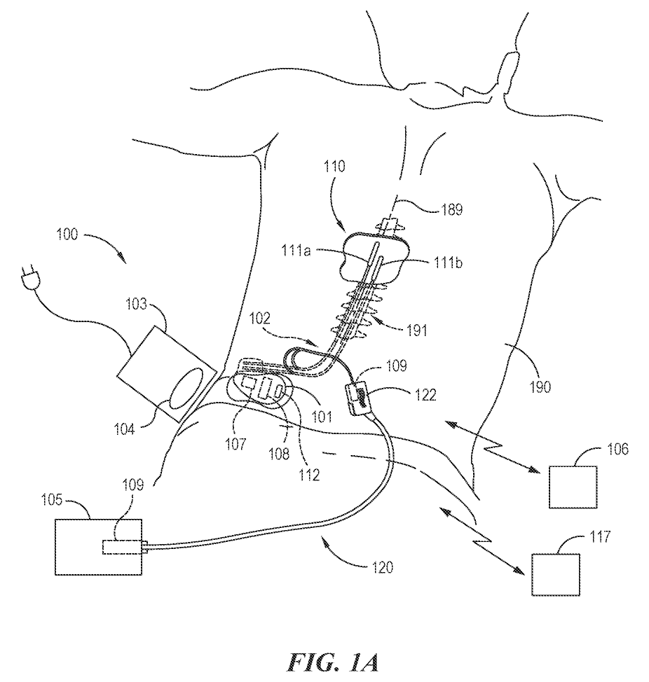

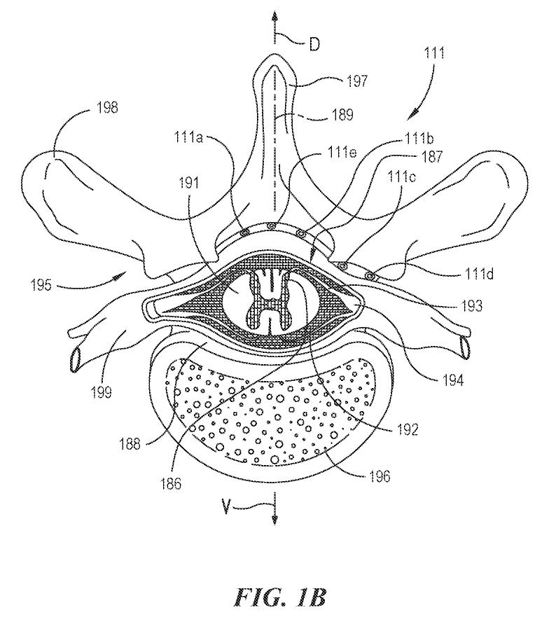

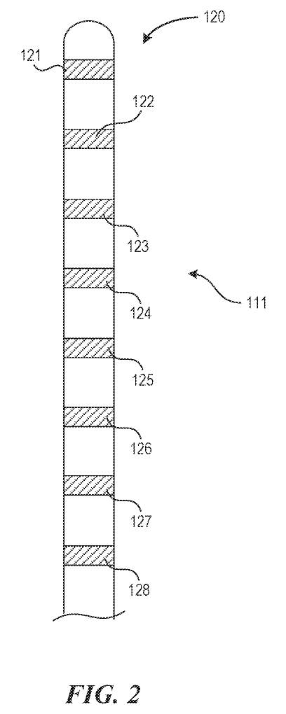

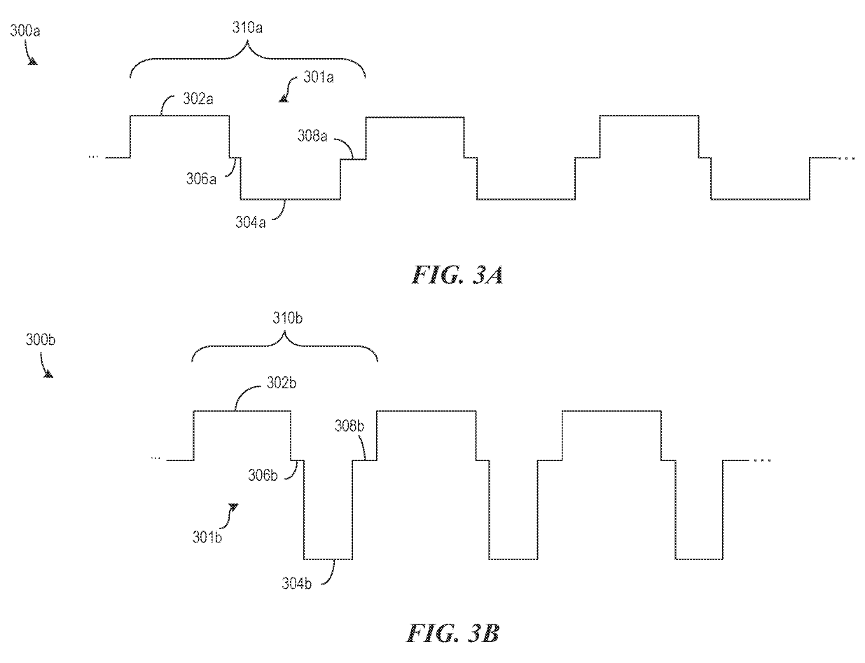

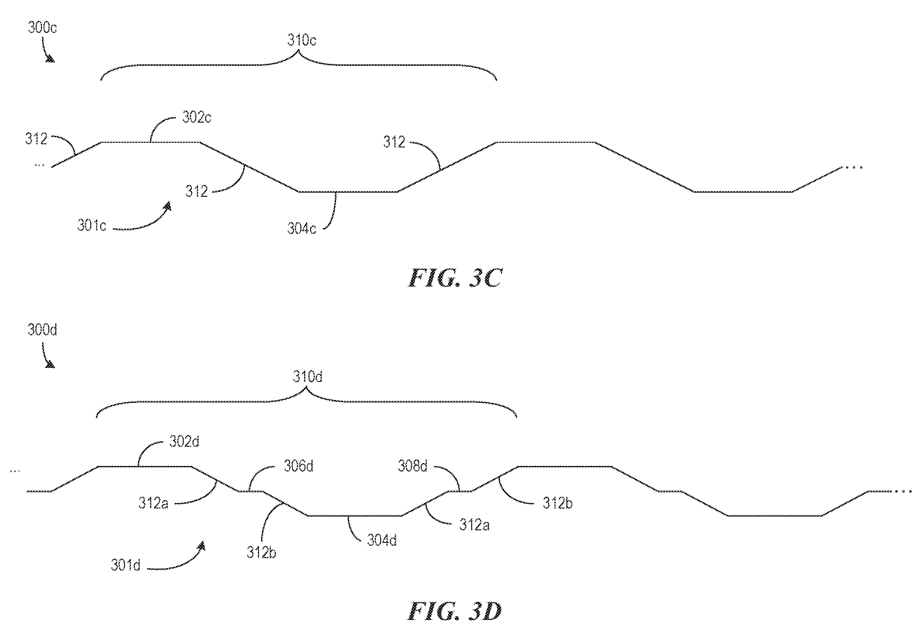

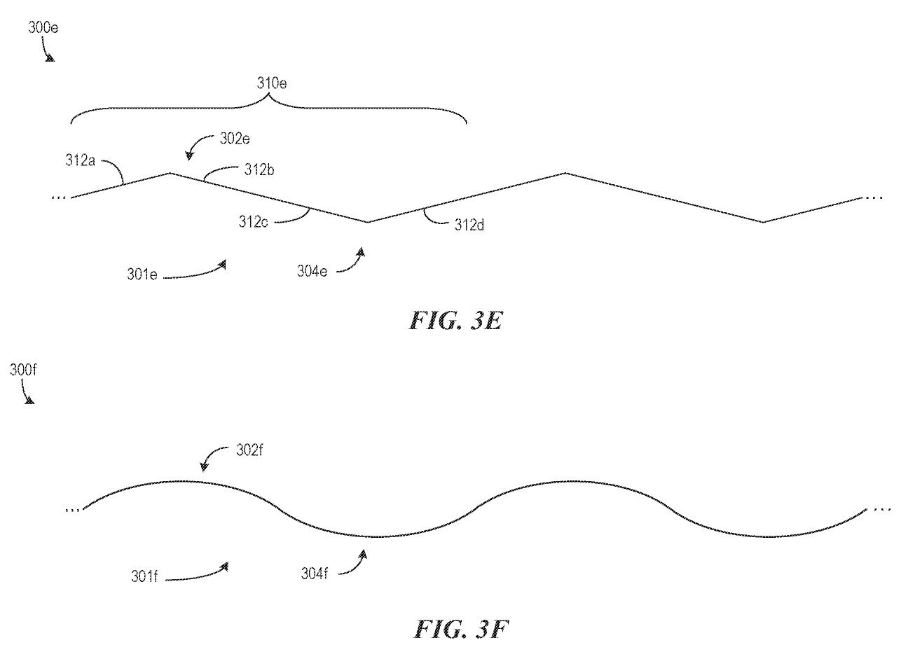

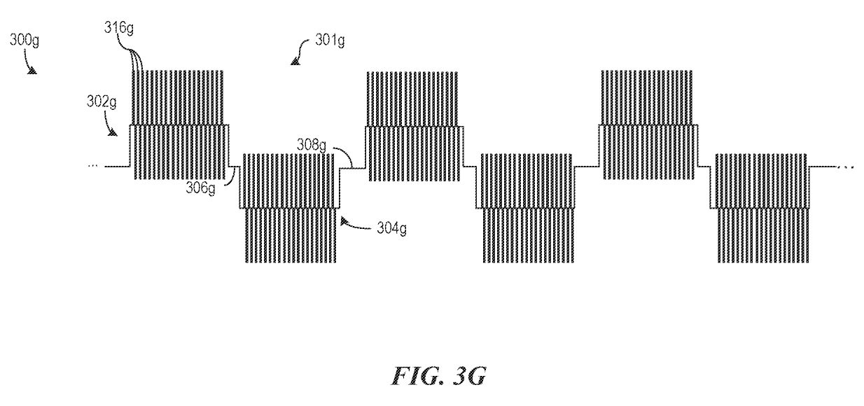

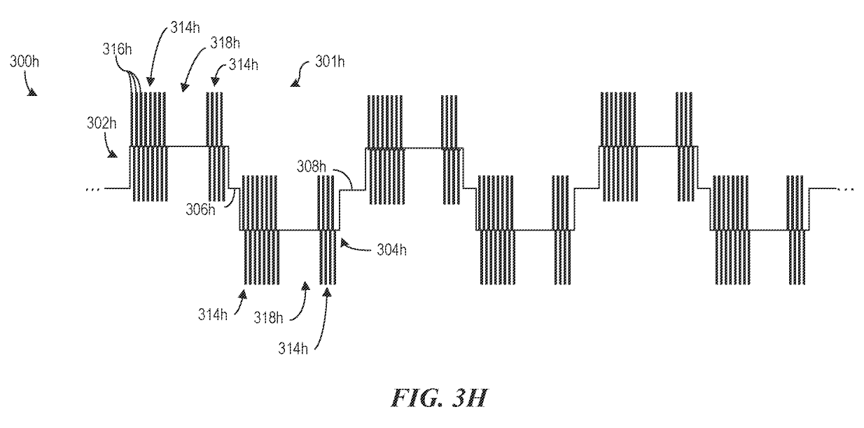

Definitions of selected terms are provided under Heading 1.0 ("Definitions"). Representative treatment systems and their characteristics are described under Heading 2.0 ("System Characteristics") with reference to FIGS. 1A, 1B and 2 . Representative electrical signals for treating patients are described under Heading 3.0 ("Representative Electrical Signals") with reference to FIGS. 3A-3H. Use of the representative electrical signals to treat ANS dysfunction and related disorders are described under Heading 4.0 (“Treating Autonomic Nervous System Dysfunction and Related Disorders”). Representative examples are described under Heading 5.0 ("Representative Examples"). The headings provided herein are for convenience only and do not interpret the scope or meaning of the claimed present technology.

### 1.0 Definitions

Unless otherwise stated, the terms "generally," "about," and "approximately" refer to values within 10% of a stated value. For example, the use of the term "about 100" refers to a range of 90 to 110, inclusive. In instances where relative terminology is used in reference to something that does not include a numerical value, the terms are given their ordinary meaning to one skilled in the art.

As used herein, the terms “therapy signal,” “electrical therapy signal,” “electrical signal,” “neuromodulation signal,” “signal,” and other associated terms are used interchangeably and generally refer to an electrical signal that can be characterized by one or more parameters, such as pulse width, amplitude, and/or frequency.

As used herein, and unless otherwise noted, the terms "modulate," "modulation," "stimulate," and "stimulation" refer generally to signals that have an inhibitory, excitatory, and/or other effect on a target neural population. Accordingly, a spinal cord "stimulator" can have an inhibitory effect or an excitatory effect on certain neural populations. Moreover, the use of the terms "suppress" and "inhibit" in relation to a therapy signal's effect on a neuron refers to a reduction in the neuron's firing rate relative to the neuron's firing rate in the absence of the therapy signal, and does not necessarily refer to a complete elimination of action potentials in the neuron. Likewise, the use of the terms “activate,” “excite,” and “upregulate” in relation to a therapy signal’s effect on a neuron refers to an increase in the neuron’s firing rate and/or a depolarization toward an action potential threshold, relative to the neuron’s firing rate and/or polarization in the absence of the therapy signal.

As used herein, the terms “autonomic nervous system dysfunction,” “ANS dysfunction,” “autonomic dysfunction,” “dysautonomia,” and the like refers to abnormal functioning of one or more nerves associated with the ANS, and can include (a) overactive and/or dominant parasympathetic activity, (b) underactive parasympathetic activity, (c) overactive and/or dominant sympathetic activity, (d) underactive sympathetic activity, (e) imbalance between parasympathetic activity and sympathetic activity, (f) abnormal combined effect of parasympathetic activity and sympathetic activity, and/or combinations of any of the foregoing.

As used herein, "proximate a spinal cord region" refers to the placement of a signal delivery element such that it can deliver electrical stimulation to a neural population associated with the spinal cord or associated nervous system structures. For example, "proximate a spinal cord region" includes, but is not limited to, the relative lead positions described and shown in FIG. 1B, as well as other positions not expressly described herein.

As used herein, the term "pulse width" refers to the width of any phase of a repeating pulse, such as the portion of a pulse at a given polarity, unless explicitly described otherwise. For example, the use of the term pulse width with respect to a signal having bi-phasic pulses can refer to the duration of an anodic pulse phase or a cathodic pulse phase. The use of the term pulse width with respect to a signal having monophasic pulses can refer to the duration of the monophasic pulse phase.

### 2.0 System Characteristics

FIG. 1A schematically illustrates a representative patient therapy system 100 for treating a patient's motor and/or other functioning, arranged relative to the general anatomy of the patient's spinal column 191. The system 100 can include a signal generator 101 (e.g., an implanted or implantable pulse generator or IPG), which may be implanted subcutaneously within a patient 190 and coupled to one or more signal delivery elements or devices 110. The signal delivery elements or devices 110 may be implanted within the patient 190, at or off the patient's spinal cord midline 189. The signal delivery elements 110 carry features for delivering therapy to the patient 190 after implantation. The signal generator 101 can be connected directly to the signal delivery devices 110, or it can be coupled to the signal delivery devices 110 via a signal link, e.g., a lead extension 102. In some embodiments, the signal delivery devices 110 can include one or more elongated lead(s) or lead body or bodies 111 (identified individually as a first lead 111a and a second lead 111b). As used herein, the terms signal delivery device, signal delivery element, lead, and/or lead body include any of a number of suitable substrates and/or supporting members that carry electrodes/devices for providing therapy signals to the patient 190. For example, the lead or leads 111 can include one or more electrodes or electrical contacts that deliver electrical signals into the patient's tissue, e.g., to provide for therapeutic relief. In some embodiments, the signal delivery elements 110 can include structures other than a lead body (e.g., a paddle) that also deliver electrical signals and/or other types of signals to the patient 190, e.g., as disclosed in U.S. Patent Application Publication No. 2018/0256892, incorporated herein by reference in its entirety. For example, paddles may be more suitable for patients with spinal cord injuries that result in scarring or other tissue damage that impedes cylindrical leads.

In some embodiments, one signal delivery device may be implanted on one side of the spinal cord midline 189, and a second signal delivery device may be implanted on the other side of the spinal cord midline 189. For example, the first and second leads 111a, 111b shown in FIG. 1A may be positioned just off the spinal cord midline 189 (e.g., about 1 mm offset) in opposing lateral directions so that the two leads 111a, 111b are spaced apart from each other by about 2 mm. In some embodiments, the leads 111 may be implanted at a vertebral level ranging from, for example, about T1 to about T12. In some embodiments, one or more signal delivery devices can be implanted at other vertebral levels, e.g., as disclosed in U.S. Patent No. 9,327,121, incorporated herein by reference in its entirety.

The signal generator 101 can transmit signals (e.g., electrical signals) to the signal delivery elements 110 that excite, upregulate, inhibit, downregulate, and/or suppress target nerves. The signal generator 101 can include a machine-readable (e.g., computer-readable or controller-readable) medium containing instructions for generating and transmitting suitable therapy signals, such as those described below with respect to FIGS. 3A–3D. The signal generator 101 and/or other elements of the system 100 can include one or more processor(s) 107, memory unit(s) 108, and/or input/output device(s) 112. Accordingly, the process of providing modulation signals, providing guidance information for positioning the signal delivery devices 110, establishing battery charging and/or discharging parameters, and/or executing other associated functions can be performed by computer-executable instructions contained by, on, or in computer-readable media located at the pulse generator 101 and/or other system components. Further, the pulse generator 101 and/or other system components may include dedicated hardware, firmware, and/or software for executing computer-executable instructions that, when executed, perform any one or more methods, processes, and/or sub-processes described in the materials incorporated herein by reference. The dedicated hardware, firmware, and/or software also serve as “means for” performing the methods, processes, and/or sub-processes described herein. The signal generator 101 can also include multiple portions, elements, and/or subsystems (e.g., for directing signals in accordance with multiple signal delivery parameters), carried in a single housing, as shown in FIG. 1A, or in multiple housings. For example, the signal generator can include some components that are implanted (e.g., a circuit that directs signals to the signal delivery device 110), and some that are not (e.g., a power source). The computer- executable instructions can be contained on one or more media that are implanted within the patient and/or positioned external to the patient, depending on the embodiment.

The signal generator 101 can also receive and respond to an input signal received from one or more sources. The input signals can direct or influence the manner in which the therapy, charging, and/or process instructions are selected, executed, updated, and/or otherwise performed. The input signals can be received from one or more sensors (e.g., an input device 112 shown schematically in FIG. 1A for purposes of illustration) that are carried by the signal generator 101 and/or distributed outside the signal generator 101 (e.g., at other patient locations) while still communicating with the signal generator 101. The sensors and/or other input devices 112 can provide inputs that depend on or reflect patient state (e.g., patient position, patient posture, and/or patient activity level), and/or inputs that are patient-independent (e.g., time). Still further details are included in U.S. Patent No. 8,355,797, incorporated herein by reference in its entirety.

In some embodiments, the signal generator 101 and/or signal delivery devices 110 can obtain power to generate the therapy signals from an external power source 103. For example, the external power source 103 can by-pass an implanted signal generator and generate a therapy signal directly at the signal delivery devices 110 (or via signal relay components). The external power source 103 can transmit power to the implanted signal generator 101 and/or directly to the signal delivery devices 110 using electromagnetic induction (e.g., RF signals). For example, the external power source 103 can include an external coil 104 that communicates with a corresponding internal coil (not shown) within the implantable signal generator 101, signal delivery devices 110, and/or a power relay component (not shown). The external power source 103 can be portable for ease of use. In embodiments without the implanted signal generator 101 and in which the external power source 103 causes a therapy signal to be generated directly at the signal delivery devices 110 or at an implanted relay device (not shown), the external power source 103 can be a wearable device that the patient wears while receiving therapy. In such embodiments, the patient only receives stimulation therapy while the wearable device is placed in an active state and is being worn by the patient. Such embodiments are generally less invasive because they generally do not require an implanted signal generator 101, nor an implanted power storage device.

In some embodiments, the signal generator 101 can obtain the power to generate therapy signals from an internal power source, in addition to or in lieu of the external power source 103. For example, the implanted signal generator 101 can include a non-rechargeable battery (e.g., a primary cell) or a rechargeable battery (e.g., a secondary cell) to provide such power. When the internal power source includes a rechargeable battery, the external power source 103 can be used to recharge the battery. The external power source 103 can in turn be recharged from a suitable power source (e.g., conventional wall power).

During at least some procedures, an external stimulator or trial modulator 105 can be coupled to the signal delivery elements 110 during an initial procedure, prior to implanting the signal generator 101. For example, a practitioner (e.g., a physician and/or a company representative) can use the trial modulator 105 to vary the modulation parameters provided to the signal delivery elements 110 in real time, and select optimal or particularly efficacious parameters. These parameters can include the location from which the electrical signals are emitted, as well as the characteristics of the electrical signals provided to the signal delivery devices 110. In some embodiments, input is collected via the external stimulator or trial modulator 105 and can be used by the clinician to help determine what parameters to vary. In a typical process, the practitioner uses a wireless connection or cable assembly 120 to temporarily connect the trial modulator 105 to the signal delivery device 110. The practitioner can test the efficacy of the signal delivery devices 110 in an initial position. The practitioner can then disconnect the cable assembly 120 if needed (e.g., at a connector 122), reposition the signal delivery devices 110, and reapply the electrical signals. This process can be performed iteratively until the practitioner obtains the desired position for the signal delivery devices 110. Optionally, the practitioner may move the partially implanted signal delivery devices 110 without disconnecting the cable assembly 120. Furthermore, in some embodiments, the iterative process of repositioning the signal delivery devices 110 and/or varying the therapy parameters may not be performed.

The signal generator 101, the lead extension 102, the trial modulator 105 and/or the connector 122 can each include a receiving element 109. Accordingly, the receiving elements 109 can be patient implantable elements, or the receiving elements 109 can be integral with an external patient treatment element, device or component (e.g., the trial modulator 105 and/or the connector 122). The receiving elements 109 can be configured to facilitate a simple coupling and decoupling procedure between the signal delivery devices 110, the lead extension 102, the pulse generator 101, the trial modulator 105 and/or the connector 122. The receiving elements 109 can be at least generally similar in structure and function to those described in U.S. Patent Application Publication No. 2011/0071593, incorporated by reference herein in its entirety.

After the signal delivery elements 110 are implanted, the patient 190 can receive therapy via signals generated by the trial modulator 105, generally for a limited period of time. During this time, the patient wears the trial modulator 105 outside the body. Assuming the trial therapy is effective or shows the promise of being effective, the practitioner then replaces the trial modulator 105 with the implanted signal generator 101, and programs the signal generator 101 with therapy programs selected based on the experience gained during the trial period. Optionally, the practitioner can also replace the signal delivery elements 110. In still further embodiments, the signal generator 101 can be implanted without first undergoing a trial period. Once the implantable signal generator 101 has been positioned within the patient 190, the therapy programs provided by the signal generator 101 can still be updated remotely via a wireless physician's programmer 117 (e.g., a physician's laptop, a physician's remote or remote device, etc.) and/or a wireless patient programmer 106 (e.g., a patient's laptop, patient's remote or remote device, etc.). Generally, the patient 190 has control over fewer parameters than does the practitioner. For example, the capability of the patient programmer 106 may be limited to starting and/or stopping the signal generator 101, selecting a pre-programmed therapy option, and/or adjusting the signal amplitude within a present amplitude range. The patient programmer 106 may be configured to accept inputs corresponding to pain relief, motor functioning and/or other variables, such as medication use. Accordingly, more generally, embodiments of the present technology include receiving patient feedback, via a sensor, that is indicative of, or otherwise corresponds to, the patient's response to the signal. Feedback includes, but is not limited to, motor, sensory, and verbal feedback. In response to the patient feedback, one or more signal parameters can be adjusted, such as frequency, pulse width, amplitude, or delivery location. In some embodiments, the patient programmer can be a network connected handheld computing device such as a smartphone, which can include a patient app that provides various functions such as remote programming, therapy selection, therapy tracking information such as current pain score or level of sensory restoration for use by the clinician and/or software in the app to optimize therapy.

FIG. 1B is a cross-sectional illustration of the spinal cord 191 and an adjacent vertebra 195 (based generally on information from Crossman and Neary, "Neuroanatomy," 1995 (published by Churchill Livingstone)), along with multiple leads 111 (shown as leads 111a-111e) implanted at representative locations. For purposes of illustration, multiple leads 111 are shown in FIG. 1B implanted in a single patient. In addition, for purposes of illustration, the leads 111 are shown as elongated leads however, leads 111 can be paddle leads. In actual use, any given patient will likely receive fewer than all the leads 111 shown in FIG. 1B.

The spinal cord 191 is situated within a vertebral foramen 188, between a ventrally located ventral body 196 and a dorsally located transverse process 198 and spinous process 197. Arrows V and D identify the ventral and dorsal directions, respectively. The spinal cord 191 itself is located within the dura mater 199, which also surrounds portions of the nerves exiting the spinal cord 191, including the ventral roots 192, dorsal roots 193, and dorsal root ganglia 194. The dorsal roots 193 enter the spinal cord 191 at the dorsal root entry region 187, and communicate with dorsal horn neurons located at the dorsal horn 186. In some embodiments, the first and second leads 111a, 111b are positioned just off the spinal cord midline 189 (e.g., about 1 mm offset) in opposing lateral directions so that the two leads 111a, 111b are spaced apart from each other by about 2 mm, as discussed above. In some embodiments, a lead or pairs of leads can be positioned at other locations, e.g., toward the outer edge of the dorsal root entry portion 187 as shown by a third lead 111c, or at the dorsal root ganglia 194, as shown by a fourth lead 111d, or approximately at the spinal cord midline 189, as shown by a fifth lead 111e.

In some embodiments the devices and systems of the present technology include features other than those described herein. For example, one lead 111 to six leads 111 can be positioned generally end-to-end at or near the patient's midline M and span vertebral levels from about T4 to about T12. In some embodiments, two, three, or four leads 111 are positioned end-to-end at or near the patient's midline from T4 to T12. In some embodiments, the leads 111 and/or other signal delivery devices can have locations other than those expressly shown herein. For example, one or more signal delivery devices can be positioned at the dorsal side of the spinal cord 191. In addition, the devices and systems of the present technology can include more than one internal stimulator and/or more than one external stimulator that can be configured for wireless stimulation, such as by using electromagnetic waves.

Several aspects of the technology are embodied in computing devices, e.g., programmed/programmable pulse generators, controllers and/or other devices. The computing devices on/in which the described technology can be implemented may include one or more central processing units, memory, input devices (e.g., input ports), output devices (e.g., display devices), storage devices, and network devices (e.g., network interfaces). The memory and storage devices are computer-readable media that may store instructions that implement the technology. In some embodiments, the computer readable media are tangible media. In some embodiments, the data structures and message structures may be stored or transmitted via an intangible data transmission medium, such as a signal on a communications link. Various suitable communications links may be used, including but not limited to a local area network and/or a wide-area network.

FIG. 2 is a partially schematic illustration of a representative lead body 111 that may be used to apply modulation to a patient in accordance with any of the foregoing embodiments. In general, the lead body 111 includes a multitude of electrodes or contacts 120. When the lead body 111 has a circular cross-sectional shape, as shown in FIG. 2, the contacts 120 can have a generally ring-type shape and can be spaced apart axially along the length of the lead body 111. In a particular embodiment, the lead body 111 can include eight contacts 120, identified individually as first, second, third . . . eighth contacts 121, 122, 123 . . . 128. In general, one or more of the contacts 120 are used to provide signals, and another one or more of the contacts 120 provide a signal return path. Accordingly, the lead body 111 can be used to deliver monopolar modulation (e.g., if the return contact is spaced apart significantly from the delivery contact), or bipolar modulation (e.g., if the return contact is positioned close to the delivery contact and in particular, at the same target neural population as the delivery contact). In still further embodiments, the pulse generator 101 (FIG. 1A) can operate as a return contact for monopolar modulation.

### 3.0 Representative Electrical Signals

FIG. 3A is a partially schematic illustration of a representative electrical therapy signal 300a used to delivery therapy in accordance with embodiments of the present technology. The therapy signal 300a includes biphasic pulses 301a repeating in a continuous manner. Each individual pulse 301a includes an anodic pulse phase 302a, a cathodic pulse phase 304a, and an interphase interval 306a separating the anodic pulse phase 302a and the cathodic pulse phase 304a. In the illustrated embodiment, the anodic pulse phase 302a and the cathodic pulse phase 304a are symmetrical (e.g., having generally equal pulse widths and generally equal and opposite amplitudes) such that individual pulses 301a are charge balanced. Individual pulses 301a are separated by an interpulse interval 308a. Together, the pulse 301a and the interpulse interval 308a define a pulse period 310a. The pulse period 310a repeats in cycles that define a frequency of the therapy signal 300a.

The therapy signal 300a can have relatively long pulse widths, such as between about 2 milliseconds and about 10 seconds. Accordingly, the anodic pulse phase 302a and the cathodic pulse phase 304a can each have a pulse width in a range of from about 2 milliseconds to about 10 seconds. In embodiments for which the pulse 301a is a monophasic pulse, the monophasic pulse phase can have a pulse width between about 2 milliseconds to about 10 seconds. In some embodiments, the therapy signal 300a can have a pulse width of between about 2 milliseconds and about 5 seconds, or between about 5 milliseconds and about 5 seconds, or between about 2 milliseconds and about 1 second, or between about 100 milliseconds and about 1 second, or between about 100 milliseconds and about 500 milliseconds, or between about 150 milliseconds and 1 second, or between about 150 milliseconds and about 750 milliseconds, or between about 150 milliseconds and about 500 milliseconds, or between about 200 milliseconds and about 1 second, or between about 200 milliseconds and about 750 milliseconds, or between about 200 milliseconds and about 500 milliseconds, or between 500 milliseconds and 1.5 seconds, or between 600 milliseconds and 1.2 seconds, or between 600 milliseconds and 1 second. Representative pulse widths include about 2 milliseconds, about 5 milliseconds, about 10 milliseconds, about 25 milliseconds, about 50 milliseconds, about 75 milliseconds, about 100 milliseconds, about 150 milliseconds, about 200 milliseconds, about 250 milliseconds, about 300 milliseconds, about 350 milliseconds, about 400 milliseconds, about 450 milliseconds, about 500 milliseconds, about 550 milliseconds, about 600 milliseconds, about 650 milliseconds, about 700 milliseconds, about 750 milliseconds, about 800 milliseconds, about 850 milliseconds, about 900 milliseconds, about 950 milliseconds, about 1 second, and/or about 2 seconds. In some embodiments, the pulse width is greater than about 2 milliseconds, greater than about 5 milliseconds, greater than about 10 milliseconds, greater than about 25 milliseconds, greater than about 50 milliseconds, greater than about 75 milliseconds, greater than about 100 milliseconds, greater than about 200 milliseconds, greater than about 300 milliseconds, greater than about 400 milliseconds, greater than about 500 milliseconds, and/or greater than about 1 second.

In the illustrated embodiment, the anodic pulse phase 302a and the cathodic pulse phase 304a have generally equal pulse widths that can offset charge build up in a signal delivery element (e.g., electrodes 120) and/or surrounding tissue. In other embodiments, and as described below with respect to FIG. 3B, the anodic pulse phase and cathodic pulse phase do not have the same pulse width. In yet other embodiments, the stimulation charge recovery is a passive process, in which a shunt resistance is connected across the active electrodes to allow for charge built up on the output and double-layer capacitances from the therapeutic pulse (e.g., the anodic pulse phase 302a) to 'bleed off'. In such embodiments, the therapy signal may be essentially a monophasic signal. As one skilled in the art will recognize, the frequency of the therapy signal 300a is based at least in part on the pulse width of the pulses 301a. For example, pulses with longer pulse widths typically (but not always) have lower frequencies. Accordingly, in some embodiments the frequency of the therapy signal 300a is less than about 100 Hz, less than about 10 Hz, less than about 5 Hz, and/or less than about 1 Hz.

The pulses 301a can have an amplitude (e.g., current amplitude or voltage amplitude) below the activation threshold of a nearby (e.g., target) neuronal population. In such embodiments, the therapy signal 300a generally does not induce an action potential in nearby neurons when it is delivered to the nearby neuronal population, and therefore generally does not generate paresthesia in the patient. Generally, the activation of neurons depends on two variables: the strength (e.g., amplitude) of the signal and the duration (e.g., pulse width) for which the signal is applied. As the duration of the signal increases, the amplitude required to induce neuronal activation decreases. Accordingly, the amplitude of the pulses 301a is inversely related to the pulse width of the pulses 301a. In some embodiments, the amplitude remains below the rheobase of the nearby neuronal population. The rheobase refers to the minimum amplitude that results in neuronal activation when the therapy signal is applied for a continuous period (e.g., a period exceeding 100 milliseconds, 200 milliseconds, 300 milliseconds, etc.). In some embodiments, the rheobase can be approximated by measuring the amplitude at which a patient exhibits the first clinically discernable effects of the signal. For example, in some embodiments, the amplitude of the pulses 301a is about 3 mA or less, such as between about 0.1 mA and about 2.5 mA or between about 0.5 mA and about 2 mA. In some embodiments, the pulses 301A can have an amplitude (e.g., current amplitude or voltage amplitude) above the activation threshold of a nearby (e.g., target) neuronal population. Depending on the relative amplitude of the signal and the type of neuron activated, the signal 300a still may not induce paresthesia in the patient, despite inducing an action potential in the target neuronal population.

In some embodiments, the pulses 310a have an amplitude that is less than an amplitude that generates an immediate clinically discernable effect (e.g., paresthesia, muscle twitching, etc.) in the patient. In such embodiments, a pulse width can be selected and various amplitudes can be iteratively tested to determine a maximum amplitude beyond which the patient begins to exhibit a clinically discernable effect. The signal can then be applied at an amplitude less than the determined maximum amplitude. Additional details regarding determining suitable signal parameters, including amplitudes, for the therapy signals described herein are described in U.S. Patent Nos. 11,819,693 and 12,133,981, the disclosures of which are incorporated by reference herein in their entireties.

FIG. 3B is a partially schematic illustration of another representative therapy signal 300b. Certain aspects of therapy signal 300b are generally similar to those described above with respect to the therapy signal 300a shown in FIG. 3A. For example, therapy signal 300b includes a pulse period 310b having a biphasic pulse 301b and an interpulse interval 308b. The pulse 301b has an anodic pulse phase 302b and a cathodic pulse phase 304b separated by an interphase interval 306b. Unlike the therapy signal 300a shown in FIG. 3A, however, the pulses 301b shown in FIG. 3B do not have symmetrical anodic pulse phases 302b and cathodic pulse phases 304b. Rather, the cathodic pulse phase 304b has a shorter pulse width and a greater amplitude than the anodic pulse phase 302b. In other embodiments, therapy signal 300b can have an anodic pulse phase 302b that has a shorter pulse width and a greater amplitude than the cathodic pulse phase 304b (e.g., a mirror image of therapy signal 300b). Regardless, the pulse width and amplitude of the cathodic pulse phase 304b can nevertheless be selected such that the total charge delivered in the anodic pulse phase 302b and the cathodic pulse phase 304b remains substantially equal to avoid having a charge build up in the electrode or the patient’s tissue. In some embodiments, the amplitude of the anodic pulse phase 302b can remain below the activation threshold that results in the first clinically discernable effect of the stimulation (and/or below the rheobase). Similarly, the larger amplitude of the cathodic pulse phase 302b also remains below the threshold of a clinically discernable effect of stimulation (and/or below the rheobase). One expected advantage of therapy signal 300b is the recovery period takes less time, meaning the duration between subsequent anodic pulse phases 302b is less and the frequency of the pulse period 310b can be higher.

FIG. 3C illustrates a representative therapy signal 300c with a ramping period 312. The ramping period 312 transitions between a maximum amplitude of the anodic pulse phase 302c and a maximum amplitude of the cathodic pulse phase 304c. In some embodiments (e.g., as described below with respect to FIG. 3E), the anodic pulse phase 302c and the cathodic pulse phase 304c can include a portion of the ramping period 312. The ramping period 312 can have a duration that is substantially equal to the pulse width of the therapy signal 300c. For example, if the anodic pulse phase 302c and the cathodic pulse phase 304c each have a pulse width of about 100 milliseconds, the ramping period 312 can have a duration of about 100 milliseconds. Accordingly, the ramping period 312 can have a duration between about 2 milliseconds and about 10 seconds. In other embodiments, the ramping period 312 is less than or greater than the pulse width of the anodic pulse phase 302c and/or the cathodic pulse phase 304c. Although the anodic pulse phase 302c and the cathodic pulse phase 304c are illustrated as symmetrical, the anodic pulse phase 302c and the cathodic pulse phase 304c can also have a configuration similar to that described above with respect to FIG. 3B, with the ramping period 312 extending therebetween.

FIG. 3D illustrates another therapy signal 300d having a non-continuous ramping period 312 between the anodic pulse phase 302d and the cathodic pulse phase 304d during pulse period 310d. The noncontinuous ramping period can include a first ramping period 312a immediately following the anodic pulse phase 302d and the cathodic pulse phase 304d, and a second ramping period 312b immediately preceding the anodic pulse phase 302d and the cathodic pulse phase 304d. The first ramping period 312a and the second ramping period 312b can be separated by an interphase interval 306d (e.g., between an anodic pulse phase 302d and cathodic pulse phase 304d within the same pulse 301d) or an interpulse interval 308d (e.g., between adjacent pulses 301d). The first ramping period 312a and the second ramping period 312b can have the same or different durations. Together, the first ramping period 312a and the second ramping period 312b can have a duration substantially equal to the pulse width of the therapy signal 301d (e.g., between about 2 milliseconds and about 10 seconds). In other embodiments, the first ramping period 312a and the second ramping period 312b together have a duration that is less than or greater than the pulse width of the therapy signal 301d.

As indicated above, some embodiments of the present technology include therapy signals having ramped, or at least partially ramped, anodic pulse phases and/or ramped, or at least partially ramped, cathodic pulse phases. For example, FIG. 3E illustrates a representative ramped therapy signal 300e in which the therapy signal 300e includes an anodic pulse phase 302e having a first ramping period 312a and a second ramping period 312b, and a cathodic pulse phase 304e having a third ramping period 312c and a fourth ramping period 312d. As described above, the anodic pulse phase 302e may have a pulse width of between about 2 milliseconds and about 10 seconds, and the cathodic pulse phase 304e may have a pulse width of between about 2 milliseconds and about 10 seconds. The second ramping period 312b can immediately follow the first ramping period 312a, as illustrated in FIG. 3E, or the second ramping period 312b can be spaced apart from the first ramping period 312a by a period of the anodic pulse phase 302a having a constant amplitude (e.g., as illustrated in FIG. 3C). Likewise, the fourth ramping period 312d can immediately follow the third ramping period 312c, as also illustrated in FIG. 3E, or the fourth ramping period 312d can be spaced apart from the third ramping period 312c by a period of the cathodic pulse phase 304a having a constant amplitude (e.g., as illustrated in FIG. 3C). In the illustrated embodiment, the second ramping phase 312b of the anodic pulse phase 302e immediately transitions into the third ramping period 312c of the cathodic pulse phase 304e. However, in other embodiments the second ramping period 312b of the anodic pulse phase 302e can be separated from the third ramping period 312c of the cathodic pulse phase 304e by an interphase interval (e.g., as illustrated in FIG. 3D). In the illustrated embodiment, the pulse period 310e is equal to the duration of the pulse 301e. However, in other embodiments respective pulses 301e can be separated by an interpulse interval (e.g., the fourth ramping period 312d can be separated from the first ramping period 312a).

FIG. 3F illustrates another representative therapy signal 300f used to deliver therapy in accordance with embodiments of the present technology. Unlike the therapy signals 300a-300e of FIGS. 3A-3E, which have a square wave form, a ramped wave form, or a combination thereof, the therapy signal 300f of FIG. 3F has a sinusoidal wave form pattern (or other non-linear pattern) comprising repeating curved pulses 301f. Each individual pulse 301f has an anodic pulse phase 302f and a cathodic pulse phase 304f. Similar to the signals 300a-300e described above, the anodic pulse phase 302f may have a pulse width between about 2 milliseconds and about 10 seconds, and the cathodic pulse phase 304f may have a pulse width between about 2 milliseconds and about 10 seconds.

Any of the signals 300a-f described above may further include offset high frequency pulses and/or bursts of high frequency pulses occurring during the anodic and/or cathodic pulse phases (e.g., pulses starting and ending at the non-zero amplitudes of the anodic and cathodic pulse phases). For example, FIG. 3G illustrates a representative signal 300g that is generally similar to the therapy signal 300a shown in FIG. 3A but further includes high frequency pulses 316g occurring during the anodic pulse phase 302g and the cathodic pulse phase 304g of the pulse 301g. As illustrated, the high frequency pulses 316g generally occur during the anodic pulse phase 302g and/or the cathodic pulse phase 304g, but are generally absent during the interphase interval 306g and the interpulse interval 308g. Although shown as occurring during both the anodic pulse phase 302g and the cathodic pulse phase 304g, in other embodiments, the high frequency pulses 316g occur only during the anodic pulse phase 302g. In yet other embodiments, the high frequency pulses 316g occur only during the cathodic pulse phase 304g.

The high frequency pulses 316g can have a frequency in a frequency range of from about 1.2 kHz and about 100 kHz. For example, the high frequency pulses 316g can have a frequency in a frequency range of from about 1.2 kHz to about 50 kHz, from about 1.2 kHz to about 25 kHz, from about 3 kHz to about 15 kHz, or from about 5 kHz to about 15 kHz. In some embodiments, the high frequency pulses 316g have a frequency of about 5 kHz, about 10 kHz, about 15 kHz, about 20 kHz, about 25 kHz, about 50 kHz, or about 100 kHz. The high frequency pulses 316g can have a pulse width in a pulse width range of from about 10 microseconds to about 333 microseconds, from about 25 microseconds to about 166 microseconds, from about 33 microseconds to about 100 microseconds, or from about 50 microseconds to about 166 microseconds. In some embodiments, such as the embodiment illustrated in FIG. 3G, the high frequency pulses 316g have an amplitude that is greater than the amplitude of the underlying anodic pulse phase 302g and/or the cathodic pulse phase 304g. In other embodiments, the high frequency pulses 316g have an amplitude that is equal to or less than the amplitude of the anodic pulse phase 302g and/or the cathodic pulse phase 304g. Further yet, although shown as bi-phasic pulses, the high frequency pulses 316g may instead be monophasic pulses. The high frequency pulses 316g can also have a ramped and/or sinusoidal shape.

The anodic pulse phase 302g may have an overall pulse width between about 2 milliseconds and about 10 seconds (not accounting for any phase change during the high frequency pulses 316g), as described in detail above for the signal 300a of FIG. 3A. Likewise, the cathodic pulse phase 304g may also have an overall pulse width between about 2 milliseconds and about 10 seconds (not accounting for any phase change during the high frequency pulses 316g).

The therapy signal 300g can be described as having a base component (e.g., base component pulses or low frequency pulses having the anodic pulse phase 302g and the cathodic pulse phase 304g) and a high frequency component (e.g., the high frequency pulses 316g). The base component may also be referred to as a low frequency component. In some embodiments, the base component and the high frequency component are a single waveform, and therefore are generally administered using the same electrodes/contacts. In other embodiments, the high frequency component is superimposed over the base component to create the therapy signal 300g.

FIG. 3H illustrates another representative signal 300h used to deliver therapy in accordance with embodiments of the present technology. The signal 300h is generally similar to the signal 300g shown in FIG. 3G, but instead of delivering high frequency pulses during the entirety of (or at least approximately the entirety of) the anodic pulse phase and the cathodic pulse phase, the signal 300h includes high frequency pulse bursts 314h (“the bursts 314h”) occurring during only portions of the anodic pulse phase 302h and the cathodic pulse phase 304h of the pulse 301h. Sequential bursts 314h are separated by a quiescent period 318h. As illustrated, the bursts 314h generally occur during the anodic pulse phase 302h and/or the cathodic pulse phase 304h, but are generally absent during the interphase interval 306h and the interpulse interval 308h. Although shown as occurring during both the anodic pulse phase 302h and the cathodic pulse phase 304h, in other embodiments, the bursts 314h occur only during the anodic pulse phase 302h. In yet other embodiments, the bursts 314h occur only during the cathodic pulse phase 304h.

The bursts 314h include one or more individual high frequency pulses 316h repeating at an intra-burst frequency in a frequency range of from about 1.2 kHz and about 100 kHz, from about 1.2 kHz to about 50 kHz, from about 1.2 kHz to about 25 kHz, from about 3 kHz to about 15 kHz, or from about 5 kHz to about 15 kHz. In some embodiments, the intra-burst frequency of the high frequency pulses 316h is about 5 kHz, about 10 kHz, about 15 kHz, about 20 kHz, about 25 kHz, about 50 kHz, or about 100 kHz. The high frequency pulses 316h can have a pulse width in a pulse width range of from about 10 microseconds to about 333 microseconds, from about 25 microseconds to about 166 microseconds, from about 33 microseconds to about 100 microseconds, or from about 50 microseconds to about 166 microseconds. In some embodiments, such as the embodiment illustrated in FIG. 3H, the high frequency pulses 316h have an amplitude that is greater than the amplitude of the underlying anodic pulse phase 302h and/or the cathodic pulse phase 304h. In other embodiments, the high frequency pulses 316h have an amplitude that is equal to or less than the amplitude of the anodic pulse phase 302h and/or the cathodic pulse phase 304h. Further yet, although shown as bi-phasic pulses, the high frequency pulses 316h may instead be monophasic pulses. The high frequency pulses 316h can also have a ramped and/or sinusoidal shape.

Each sequential burst of the high frequency pulse bursts 314h can include the same or a different number of individual high frequency pulses 316h, compared to the preceding burst 314h. For example, in the illustrated embodiment, the bursts 314h are shown as having either eight or four individual high frequency pulses 316h. In other embodiments, other numbers of high frequency pulses 316h can be delivered during the bursts 314h.

The anodic pulse phase 302h may have an overall pulse width between about 2 milliseconds and about 10 seconds (not accounting for any phase change during the bursts 314h), as described in detail above for the signal 300a of FIG. 3A. Likewise, the cathodic pulse phase 304h may also have an overall pulse width between about 2 milliseconds and about 10 seconds (not accounting for any phase change during the bursts 314h).

As with the therapy signal 300g of FIG. 3G, the therapy signal 300h of FIG. 3H can also be described as having a base component (e.g., the anodic pulse phase 302h and the cathodic pulse phase 304h) and a high frequency component (e.g., burst 314h of high frequency pulses 316h). In some embodiments, the base component and the high frequency component are a single waveform. In other embodiments, the high frequency component is superimposed over the base component to create the therapy signal 300h.

In some embodiments, the electrical signals 300a-h shown and described with reference to FIGS. 3A-3H do not produce paresthesia when delivered to the patient, and can therefore be referred to as “non-paresthesia producing electrical signals” or “paresthesia-free electrical signals.” For example, the electrical signals 300a-h can be delivered at an amplitude below an activation threshold of certain neurons, and/or below a perception threshold of the patient. This is independent of whether the patient is suffering from a disease-induced paresthesia (e.g., paresthesia or tingling caused by a neuropathy as opposed to paresthesia induced by the electrical signal). Accordingly, in some embodiments a paresthesia-free signal may be administered to a patient suffering from disease-induced paresthesia. Indeed, in some embodiments the electrical signals provided herein can even reduce disease-induced paresthesia, in addition to addressing ANS dysfunction. Thus, in some embodiments, the electrical signals (1) do not induce paresthesia in the patient, (2) reduce naturally occurring paresthesia, and (3) address ANS dysfunction.

### 4.0 Treating Autonomic Nervous System Dysfunction and Related Disorders

ANS dysfunction can be caused by or otherwise associated with an underlying disease (e.g., secondary ANS dysfunction). For example, conditions such as diabetes (e.g., Type 2 diabetes), Parkinson’s disease, cancer, autoimmune disease, neuropsychiatric disorders, heart disease, heart failure, renal disease, and others can cause or contribute to ANS dysfunction. This ANS dysfunction itself can cause and/or contribute to various symptoms associated with the underlying disease. ANS dysfunction can also occur on its own without a known underlying cause (e.g., primary or idiopathic ANS dysfunction). Representative primary ANS disorders include, but are not limited to orthostatic hypotension, postprandial hypotension, multiple system atrophy, pure autonomic failure, afferent baroreflex failure, and familial dysautonomia.

In some embodiments, ANS dysfunction may cause or at least contribute to organ dysfunction. Representative organs that can have ANS-induced dysfunction include, but are not limited to, the brain, the heart (e.g., reduction in cardiac output, decreased or increased contraction force, decreased or increased heart rate, arrhythmias, etc.); blood vessels (e.g., abnormally increased or decreased dilation or constriction, etc.); pancreas (e.g., decreased insulin production or section, increased glucagon production or secretion, etc.), stomach/intestine/GI-tract (e.g., decreased or increased motility and tone, etc.); liver (e.g., decreased glucose uptake and/or storage), kidneys, spleen, gallbladder, ureter, urinary bladder, sex organs, skin, and others.

The present technology is expected to be useful in treating both primary and secondary ANS dysfunction, e.g., to treat organ dysfunction caused by primary and/or secondary ANS. For example, any of the electrical signals 300a-h shown and described with reference to FIGS. 3A-3H can be delivered to a patient having primary ANS dysfunction, secondary ANS dysfunction, or both primary and secondary ANS dysfunction. Without intending to be bound by theory, it is expected that the electrical signals described herein will modulate the patient’s nervous system in a manner that normalizes activity of the parasympathetic nervous system, normalizes activity of the sympathetic nervous system, and/or normalizes a balance of activity between the parasympathetic and sympathetic nervous systems. For example, the electrical signals may provide or increase a level of homeostasis or equilibrium of the ANS, and/or alter the output of the ANS. In turn, it is expected that the normalization of the patient’s ANS activity will improve patient symptoms associated with the ANS dysfunction. For example, the electrical signals may reduce organ dysfunction associated with the ANS dysfunction. This may include increasing organ output, decreasing organ output, and/or producing a normalized organ output. Accordingly, the electrical signals described herein can be used to address any of the ANS-related disorders described herein (primary or secondary), and/or dysfunction in any of the organs identified herein.

Without intending to be bound by theory, one possible mechanism of action underlying the reduction in ANS dysfunction that may be achieved using the electrical signals 300a-h of FIGS. 3A-3H includes suppressing sympathetic output. More specifically, the electrical signals herein may suppress (e.g., directly suppress) certain neurons in the spinal cord (e.g., sympathetic interneurons) by directly blocking these neurons from generating action potentials. Additionally or alternative, the electrical signals may indirectly suppress sympathetic neurons by activating inhibitory interneurons. Regardless, this reduction in sympathetic outputs can impact various autonomic functions, such as reducing vasoconstriction caused by efferent signals coming from sympathetic neurons in the spinal cord, reducing drive into the adrenal cortex, medulla, and other structures to in turn reduce release of cortisol and/or various neurotransmitters (e.g., epinephrine, norepinephrine, etc.) that increase insulin resistance, and others. The foregoing mechanisms are provided by way of example only—the present technology may operate via other mechanisms in addition to or in lieu of the mechanisms discussed herein, and therefore embodiments of the present technology are not limited to a particular mechanism of action unless clearly stated otherwise.

In at least some embodiments, the electrical signal can be applied to the patient’s spinal cord region via a signal delivery device implanted at a particular vertebral level associated with the organ of interest. For example, the electrical signal can be applied to upper thoracic vertebral levels (e.g., T1-T8, T1-T4, etc.) to address cardiac and/or pulmonary autonomic system deficits, and mid-to lower thoracic vertebral levels to address gastrointestinal, liver, and/or pancreatic autonomic system deficits (e.g., T6-T12, T8-T12, etc.). In other embodiments, the electrical signal can be applied to cervical levels of the spinal cord (e.g., C3-C5) to address organs associated not only with that vertebral level, but also with vertebral levels below it. Further details of particular vertebral levels and associated organs are described in U.S. Patent No. 8,170,675, incorporated by reference herein.

In cases of secondary ANS, the electrical signals described herein can also be used to treat the underlying disease or condition that is causing or contributing to the ANS dysfunction. For example, the electrical signals 300a-h can be used to treat diabetes (e.g., Type 2), Parkinson’s disease, cancer, autoimmune disease, neuropsychiatric disorders, obesity, heart disease, heart failure, and the like. The electrical signals 300a-h may treat these diseases by reducing ANS dysfunction (e.g., by normalizing ANS activity), and/or via other mechanisms unrelated to reducing ANS dysfunction. Indeed, in some embodiments the electrical signals may both (1) reduce ANS dysfunction associated with these diseases, and (2) address non-ANS related components of the patient’s disease. For example, the electrical signals described herein may be useful in treating patient pain and/or treating disease-induced sensory loss in addition to treating ANS dysfunction, as described in U.S. Patent No. 11,819,693 and International Patent Application No. PCT/US2024/028654, the disclosures of which are incorporated by reference herein in their entireties.

As a particular non-limiting example, in response to a patient being diagnosed with or otherwise having diabetes, a user (e.g., physician or other healthcare provider) can implant a signal delivery device proximate a spinal cord region in the patient and program a signal generator to generate and deliver any of the electrical signals described herein to the patient, such as an electrical signal having a pulse width within a pulse width range of from about 2 milliseconds to about 10 seconds. The electrical signal may treat the patient’s diabetes by normalizing activity of the patient’s parasympathetic nervous system, normalizing activity of the patient’s sympathetic nervous system, and/or normalizing a balance of activity between the patient’s parasympathetic and sympathetic nervous systems. In turn, this may normalize or improve glycemic control (e.g., regulation of the patient’s blood glucose levels) e.g., by normalizing pancreatic functioning, increasing uptake of blood glucose from blood, increasing production and/or secretion of insulin, decreasing production and/or secretion of glucagon, etc.

At the same time, the electrical signal may also address one or more additional aspects of the patient’s diabetes. For example, many patients with diabetes may also have diabetic neuropathy, which can cause pain, abnormal sensation, numbness, and/or tingling, often in the patient’s lower extremities. The electrical signals described herein may also treat/address the patient’s diabetic neuropathy by reducing and/or eliminating pain, improving and/or restoring sensation, and/or reducing or eliminating tingling associated with the diabetic neuropathy. In many embodiments, this may be in addition to (e.g., independent of) normalizing and/or improving glycemic control in the patient. Accordingly, without intending to be bound by theory, the present technology is expected to be particularly useful in that it can address multiple pathologies using a single therapy. Indeed, in some embodiments multiple pathologies can be addressed using the same electrical signal applied to the same vertebral location (e.g., without having to reprogram the signal generator and/or move the implanted signal delivery device). For example, in the foregoing example, an electrical signal having a pulse width of between about 2 milliseconds and 10 seconds can be applied to the patient’s spinal cord at a vertebral level of between T1 and T12 to both improve glycemic control and address one or more symptoms of diabetic neuropathy.

The present technology therefore also includes methods of treating patients with ANS dysfunction. The methods can include programming a signal generator (e.g., the signal generator 101 of FIG. 1A) to generate and deliver any of the electrical therapy signals described herein to the patient’s spinal cord. In some embodiments, the programming operation is performed at least partially in response to the patient having or being diagnosed with ANS dysfunction. Without intending to be bound by theory, the therapy signal can treat the patient’s ANS dysfunction as described throughout this Detailed Description.

### 5.0 CONCLUSION

From the foregoing, it will be appreciated that specific embodiments of the disclosed technology have been described herein for purposes of illustration, but that various modifications may be made without deviating from the technology. For example, therapy signals described herein can be delivered at combinations of parameter values within the foregoing ranges at values that are not expressly disclosed herein. Certain aspects of the technology described in the context of particular embodiments may be combined or eliminated in other embodiments. For example, the therapy signal can be monophasic with a passive charge elimination phase. In some embodiments, the foregoing techniques can be used to address patient deficits other than sensation loss, pain, or abnormal sensations. Further, while advantages associated with certain embodiments of the disclosed technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

The use of "and/or," as in “A and/or B” refers to A alone, B alone, and both A and B. Additionally, the term "comprising" is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Where a range of values is provided, it is understood that each intervening value, to the tenth of the unit of the lower limit unless the context clearly dictates otherwise, to between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Each of the patents and patent application publications referenced herein are incorporated by reference in their entireties and for all purposes. However, to the extent any patent or patent application publication conflicts with the present disclosure, the present disclosure controls.

What is claimed is:

1. A patient treatment system for treating diabetes, the system comprising:
an implantable signal delivery device postionable proximate a patient’s spinal cord region; and
a signal generator having a controller programmed with instructions that, when executed, cause the signal generator to:
deliver an electrical signal to the patient’s spinal cord region via the implantable signal delivery device,
wherein the electrical signal has a pulse width in a pulse width range of from about 2 milliseconds to about 10 seconds, and
wherein the electrical signal treats the patient’s diabetes by normalizing activity of the patient’s parasympathetic nervous system, normalizing activity of the patient’s sympathetic nervous system, and/or normalizing a balance of activity between the patient’s parasympathetic and sympathetic nervous systems.

2. The system of claim 1 wherein the electrical signal improves glycemic control.

3. The system of claim 1 wherein the electrical signal improves glycemic control by decreasing tissue resistance to insulin.

4. The system of claim 1 wherein the electrical signal improves glycemic control by increasing glucose uptake and/or storage in the patient’s liver.

5. The system of claim 1 wherein the electrical signal addresses one or more symptoms of diabetic neuropathy in addition to improving glycemic control.

6. The system of claim 1 wherein the one or more symptoms of diabetic neuropathy addressed by the electrical signal include pain.

7. The system of claim 1 wherein the one or more symptoms of diabetic neuropathy addressed by the electrical signal include loss of sensation.

8. The system of claim 1 wherein the pulse width range is from 100 milliseconds to 1 second.

9. The system of claim 1 wherein the pulse width range is from 150 milliseconds to 500 milliseconds.

10. The system of claim 1 wherein the pulse width range is from 600 milliseconds to 1 second.

11. The system of claim 1 wherein the pulse width range is from 200 milliseconds to 750 milliseconds.

12. The system of claim 1 wherein the electrical signal is a paresthesia-free electrical signal.
